(19) United States
(12) Patent Application Publication
(10) Pub. No.: US 2026/0341426 A1
(43) Pub. Date: Oct. 29, 2026

(54) SYSTEM AND METHOD FOR GENERATING DUE DILIGENCE QUESTIONS FOR PROCUREMENT IN THE MEDICAL FIELD

(71) Applicant: 12258706 Canada Inc., DBA Med Reddie, Saint John (CA)

(72) Inventors: Kara LEBLANC, Saint John (CA); Erik HUEBNER, Waterloo (CA); Steven FYKE, Waterloo (CA)

(21) Appl. No.: 19/654,897

(22) Filed: Apr. 22, 2026

Related U.S. Application Data
(60) Provisional application No. 63/792,983, filed on Apr. 23, 2025.

(51) Int. Cl. G16H 10/20; G16H 15/00; G16H 70/20

(52) U.S. Cl. CPC G16H 10/20; G16H 15/00; G16H 70/20

(57) ABSTRACT

Methods and system for improving the procurement process in the medical field.

### CROSS-REFERENCE TO OTHER APPLICATIONS

The disclosure claims priority from U.S. Provisional Application No. 63/792,983 filed Apr. 23, 2025 which is hereby incorporated by reference.

### FIELD

The disclosure is generally directed at the procurement process and, more specifically, at a system and method for generating due diligence questions for procurement in the medical field.

### BACKGROUND

Traditional approaches to medical procurement rely heavily on static checklists, manual input from subject matter experts, and historical product familiarity. This often results in biased request for proposals (RFPs) that unintentionally favor incumbent vendors and fail to reflect the latest clinical innovations. These approaches are typically resource-intensive, reliant on outdated knowledge, require significant human effort to update question banks, review large volumes of literature, and are often misaligned with evolving procurement frameworks that prioritize patient outcomes and long-term value.

Value-based procurement (VBP) in healthcare fundamentally shifts the focus from acquiring the lowest-priced, or most familiar solutions to prioritizing overall value or other factors such as clinical outcomes, patient experience, and cost-effectiveness. This paradigm promotes continuous improvement, transparency, and encourages the adoption of solutions that enhance care delivery.

Therefore, there is provided a novel system and method for generating due diligence questions for procurement in the medical field that addresses disadvantages in current solutions.

### SUMMARY

The disclosure is directed at a method and system for generating value-based due diligence questions (DDQs) and requirement driven questions tailored to medical products or services. In one embodiment, the disclosure integrates at least one of curated databases of peer-reviewed journal research; proprietary gold-standard DDQ samples reviewed by domain experts; and/or semantic search and vector embedding models and/or large language models.

In one aspect of the disclosure, there is provided A method for generating due diligence questions for procurement in the medical field including receiving a set of summaries of journal articles associated with a medical topic; receiving a set of previously generated due diligence questions; passing the set of summaries and the set of previously generated due diligence questions through a due diligence large language model (LLM); receiving a set of due diligence questions from the due diligence LLM; and generating a request for proposal (RFP) questionnaire based on the set of due diligence questions.

In another aspect, receiving a set of summaries of journal articles includes retrieving a set of journal articles; and passing the set of journal articles through a document summary LLM to generate the set of summaries. In a further aspect, the method includes associating a list of predefined or emerging medical categories to the set of journal articles; determining relevance of journal articles to the list of predefined or emerging medical product categories; and updating due diligence question generation logic based on determined relevance. In yet another aspect, the method includes identifying new product or service categories for the list of predetermined or emerging medical categories. In yet a further aspect, identifying new product or service categories includes parsing the set of journal articles to determine new medical terms within the set of journal articles; determining a number of times the new medical terms exist within the set of journal articles; determining if the number of times the new medical terms exist passes a predetermined threshold; and adding the new medical terms that pass the threshold to the list of predetermined or emerging medical categories.

In an aspect, the method further includes refining the set of due diligence questions by passing them through the due diligence LLM to enhance clarity and remove bias.

In yet another aspect of the disclosure, there is provided a system for generating a request for proposal (RFP) questionnaire for a medical topic including a RFP generation module including a due diligence large language model (LLM); a journal article summary module including a document summary LLM; a semantic encoder model for document embeddings; a processor for communicating with the due diligence LLM and the document summary LLM; wherein the processor passes a set of journal articles associated with the medical topic to the document summary LLM to generate a set of summaries; wherein the processor passes the set of summaries and a set of previously generated due diligence questions to the due diligence LLM to generate a set of RFP questions. In another aspect, the system includes a user interface for inputting organization-specific data and receiving customized outputs. In a further aspect, the system further includes a comparison module for comparing the set of summaries with the set of previously generated due diligence questions.

In another aspect, the system includes an association module for associating the set of journal articles or set of summaries with a set of the medical topics. In yet another aspect, the system includes an analytics module for processing and providing analytical insight to users. In yet a further aspect, the system includes a new topic generation module for generating new medical topics. In another aspect, the due diligence LLM and the document summary LLM are a single LLM.

### BRIEF DESCRIPTION OF THE DRAWINGS

The disclosure will be better understood with reference to the attached drawings, in which:

FIG. 1A is a schematic diagram of a system for generating due diligence questions for medical procurement in an operational environment;

FIG. 1B is a schematic diagram of a set of local databases;

FIG. 1C is a schematic diagram of a set of modules for use in generating due diligence questions for medical procurement in an operational environment;

FIG. 2 is a flowchart showing a method of generating due diligence questions for medical procurement in an operational environment; and

FIG. 3 is a flowchart showing a method of generating journal article summaries.

### DETAILED DESCRIPTION OF THE EMBODIMENTS

The disclosure is directed at a system and method of generating due diligence questions for product or service procurement, such as but not limited to, medical procurement. In one embodiment, the disclosure uses large language models (LLMs).

In the following, for ease of understanding, certain terms and acronyms are now defined. The term “due diligence question (DDQ) is defined as a question used in procurement processes to evaluate vendors or solutions against predefined technical, clinical, regulatory, or financial criteria. The term “value-based procurement (VBP)” is defined as a healthcare procurement methodology focused on selecting solutions based on total value, including clinical outcomes, patient safety, long-term cost efficiency, and innovation potential, rather than just lowest purchase price. A “large language model (LLM)” is defined as a machine learning model trained on large text to generate, summarize and interpret human language. In embodiments, LLMs may be used to generate and refine procurement questions. The term “semantic encoder” is defined as a model that converts natural language into numerical vector embeddings, allowing for similarity-based searches. One example of a semantic encoder includes, but is not limited to, a text-embedding-004.

The term “embedding” is defined as a numerical representation of text that captures semantic meaning, enabling comparison between documents, prompts, and queries in high-dimensional space. The term “feedback loop” is defined as a mechanism by which user edits, inputs, or approvals are stored, weighted, and used to improve future model outputs through either fine-tuning or adaptive prompting.

The term “tenant or tenant-specific” is defined as a distinct organizational user group (e.g., a hospital, health authority, or clinic network) operating independently within the system with isolated data and optionally personalized model behavior. The term “gold-standard DDQs” is defined as a set of expert-reviewed due diligence questions used as high-quality reference samples for training, validation, and generation purposes. The term “emerging category detection” is defined as a system capability that uses clustering and trend analysis in scientific literature and user behavior to identify and define new product or service categories not previously cataloged.

The term “anonymized analytics” is defined as aggregated usage insights derived from user behavior that do not reveal any individual or organizational identity, ensuring privacy while enabling market intelligence. The term “prompt adaptation” is defined as a process of modifying the prompts used in LLM interactions based on user feedback or observed performance to improve relevance and output quality.

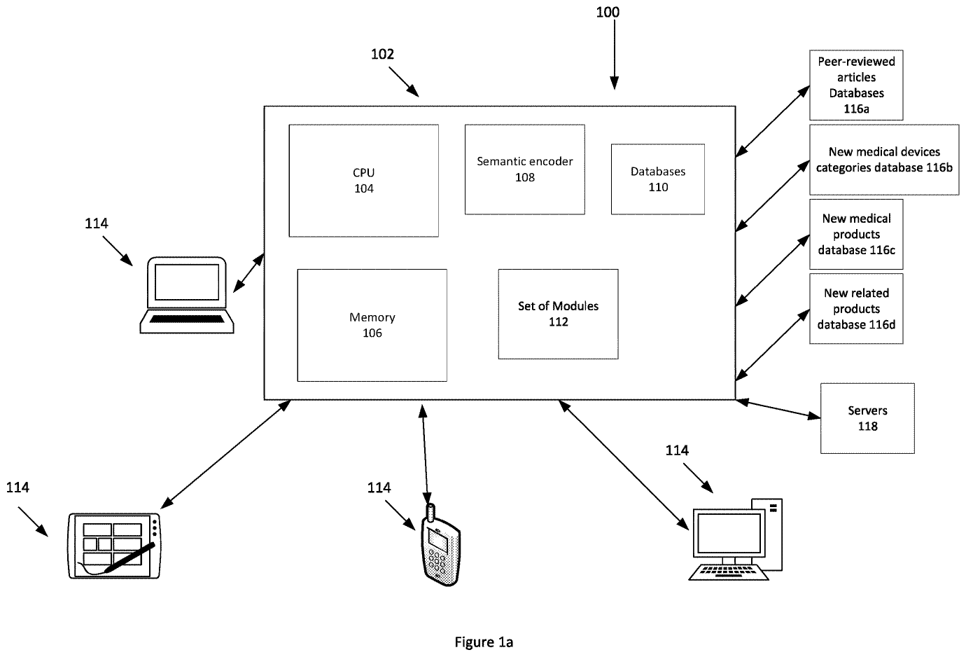

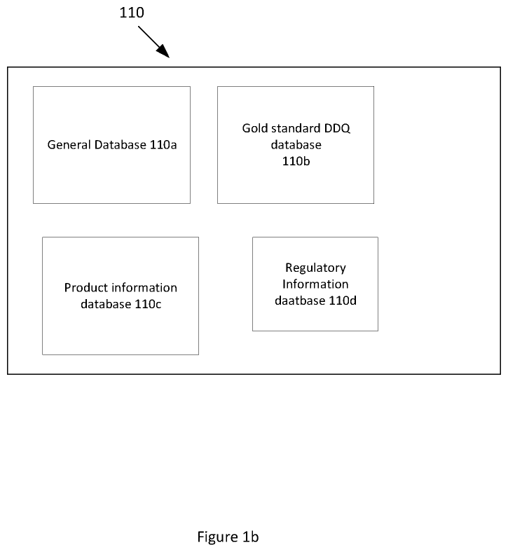

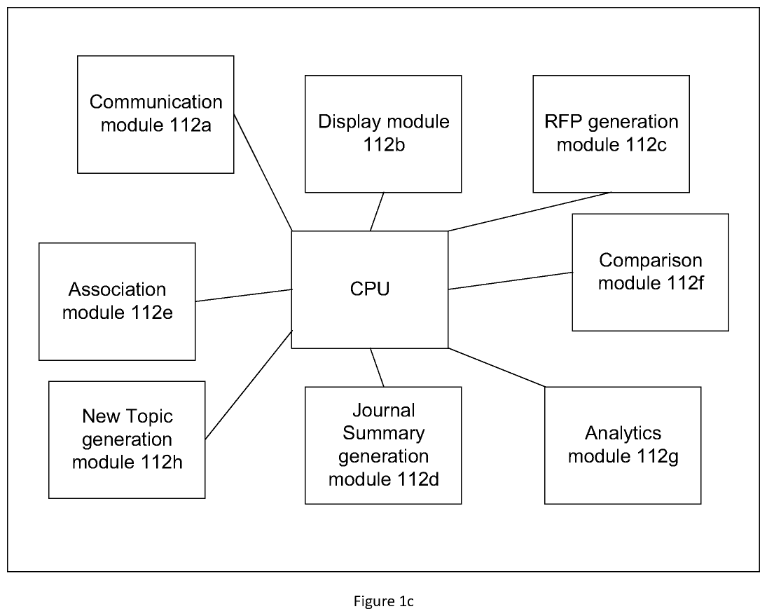

Turning to FIG. 1A, a schematic diagram of a system for generating due diligence questions for a procurement process, such as, but not limited to, a medical procurement process, is shown in an operational environment.

The system 100, which may be stored in a server 102, includes a central processing unit (CPU) 104 or processor, memory 106 (such as random access memory (RAM)), a semantic encoder 108, a set of databases 110 and a set of modules 112. As schematically shown in FIG. 1B, in one embodiment, the set of databases 110 includes a general database 110a, a gold standard DDQ database 110b, a product information database 110c and a regulatory information database 110d. In other embodiments, the set of databases 110 may include other types of databases, such as but not limited to, a vendor information database, a patient information database and the like. The semantic encoder 108 converts natural language queries and documents into embeddings where the embeddings are then used by the system to generate due diligence questions and/or a request for proposal (RFP) document.

Users (represented as communication devices 114) interact with the system 100 to transmit and receive digital data relating to a request for a set of due diligence or RFP questions. In some embodiments, the user inputs information relating to the RFP request, such as the topic relating to the RFP request, a number of desired due diligence questions and/or the type of due diligence questions and receives results from the system 100. It is understood that other inputs may be possible.

One advantage of the disclosure is that the system 100 can process a wide range of input data to tailor DDQ generation to specific use cases. These inputs may include, but are not limited to, medical product or service category (existing or new); medical product or service solution details including product specifications and clinical outcomes; question subcategory or domain (e.g., compliance, technical requirements); user-uploaded DDQs; vendor data; certifications and/or clinical goals.

In one embodiment, the users 114 communicate with the system 100 over a network, either wired or wireless, as will be described in more detail below. The network may also be the Internet.

The system may also be connected to external databases or data sources 116 to transmit and/or retrieve information from the databases 116 or to external servers 118 to request different tasks to be performed. Alternatively, system 100 may also communicate with the external servers to retrieve digital information or data that has been processed by the external server or servers 118. In one embodiment, the external server 118 may store or house a LLM that may be used by the system 100 to assist in generating due diligence questions for procurement. In one embodiment, an external server 118 may house an LLM that summarizes scientific findings. In another embodiment, an external server 118 may house an LLM such as, but not limited to, Gemini, which combines scientific findings with previously defined gold standard DDQ examples or questions.

Examples of external databases or data sources 116 include, but are not limited to, a peer-reviewed articles database 116a, a new medical devices categories database 116b, a new medical products database 116c and a new related products database 116d. The databases 116 may also be associated with other information if the procurement process is for non-medical products or services. The data sources may be seen as storing structured and unstructured digital content that supports due diligence question generation and refinement. In some embodiments, the peer-reviewed articles database 116a may store articles that are collected from public medical research databases. The articles may be constantly updated within the database whereby a processor associated with the database 116a automatically retrieves articles from other medical databases. When the information is retrieved by system 100, the system 100 extracts titles, abstracts, and keywords from the retrieved articles. Other data sources may include, but are not limited to, an industry white paper database that includes documents that are retrieved from verified open-source documents and technical reviews; a gold standard DDQ database that stored proprietary, curated list of DDQs aligned to VBP frameworks that may be organized by product/service category and reviewed by healthcare procurement experts; and a experts opinions database that stores DDQ documents that have been reviewed by experts. The DDQ documents within the experts opinions database may be reviewed by the experts to review the questions, verify fairness, confirm that no brand names have been included, ensure relevance, and provide edits and revised questions and feedback back to further train and improve the model or system of the disclosure. The external database or data source may also include user edits, feedback and historical data database that notes changes in languages or intent of generated questions to act as a feedback mechanism to understand company or user specific preferences around question phrasing or required level of detail.

Turning to FIG. 1C, a schematic diagram of a set of modules for use in the system for generating due diligence questions for a procurement process is shown. In the current embodiment, the set of modules 112 are in communication with the CPU 104 that may coordinate communication between the modules 112 or may provide signals, such as in the form of instructions, to the modules to perform their functionality to assist with the method of the disclosure. As shown in FIG. 1C, in the current embodiment, the set of modules 112 includes a communication module 112a, a display module 112b, a RFP generation module 112c, a journal article summary generation module 112d, an association module 112e, a comparison module 112f, an analytics module 112g and a new topic generation module 112h.

Communication module 112a enables communication between the system 100 and the user communication devices 114, the external databases 116 and/or the external servers 118. Communication may be performed using known communication protocols. In some embodiments, the communications may be encrypted in order to protect the information that is being transmitted between the system 100 and the other entities. Display module 112b provides the functionality to generate displays or screens that can be transmitted to the user communication devices 114 for display on the different communication devices.

RFP generation module 112c provides the functionality to process the digital information that is received or retrieved by the system to process the digital information and generate a set of due diligence questions relevant to the request from a user. The set of due diligence questions may then be formatted into an RFP document (either by the RFP generation module or the display module) for display on the communication device associated with the user requesting the RFP. The journal article summary generation module 112d processes received journal articles to generate summaries of the articles. In some embodiments, the summaries may be generated by the module 112d and, in other embodiments, the journal article summary generation module 112d may communicate with one of the external servers 118 to perform or generate the article summaries which are then transmitted back to and received by the system 100, such as via the journal article summary generation module 112d. In some embodiments, the journal summary generation module 112d may be seen as or include a summarization LLM that extracts clinical insights from journal article content. In some embodiments, the summarization LLM may be Gemini Flash.

The association module 112e provides the functionality to associate the retrieved journal articles and/or the generated journal article summaries with a list or set of medical topics, such as emerging medical topics, medical services or medical products (generally referred to as medical topics). This association may improve the RFP or due diligence question generation process by continuously associating medical topics and journal articles to increase the information for processing when generating due diligence questions. The association module 112e may also determine a relevance between the journal articles and any of the associated set of medical topics to confirm that the article and the medical topic should be associated. The comparison module 112f performs the functionality to compare journal article summaries with a set of previously generated due diligence questions to determine if the previously generated questions are relevant or should be used in a current RFP request. The comparison module 112f may also update the set of previously generated due diligence questions in response to the journal article summaries comparison. In some embodiments, the comparison module 112f may be seen as a question generation/refinement LLM that produces and refines DDQs, removing brand bias and increasing clarity. In some embodiments, the generation/refinement LLM may be Gemini Flash. It is understood that in some embodiments, the different LLMs that are used by the disclosure may be implemented in a single LLM rather than different individual LLMs.

The analytics module 112g provides the functionality to provide insights to users, such as suppliers, based on aggregated, anonymized platform activity. By monitoring procurement user behavior, such as search trends, product category interest, question edits, and upload patterns, the analytics module 112g can detect and synthesize demand signals across the operational environment. In some embodiments, the analytics module 112g may flag emerging product or new categories or underrepresented feature combinations as indicators of potential market gaps. The analytics module 112g may also be configured to generate aggregated insights for suppliers based on anonymized user interactions, including product category searches, uploaded procurement plans, and engagement with literature-linked metadata. In other embodiments, the analytics module 112g provides the functionality to identify demand signals by analyzing co-occurrence patterns between product features, clinical outcomes, and keyword activity across multiple users. The new topic generation module 112h provides the functionality to generate new medical topics that can be selected by a user and recognized by the system when requesting RFP generation.

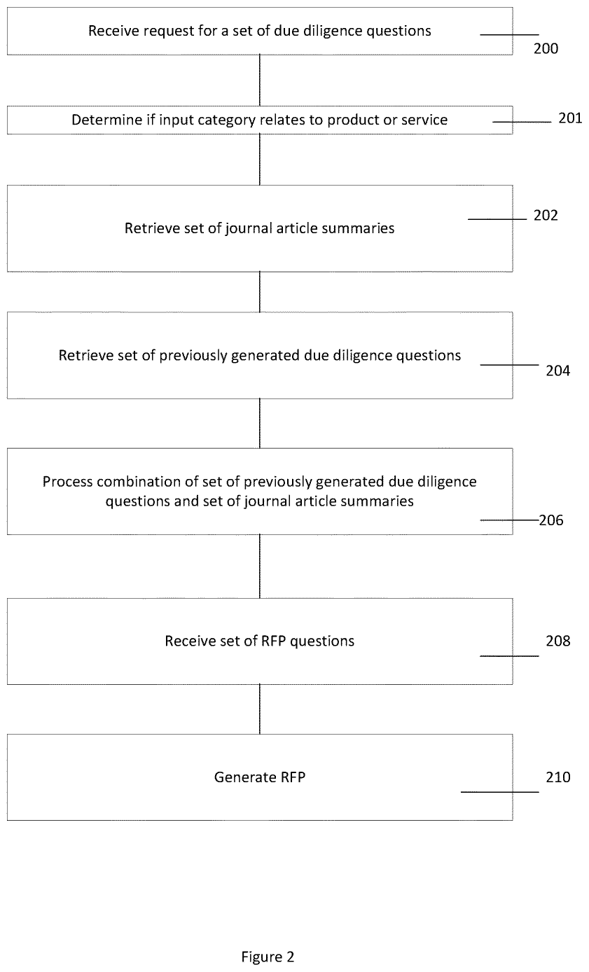

Turning to FIG. 2, a flowchart showing a method of generating a set of due diligence questions for use in creating a request for proposal (RFP) for a procurement process is shown. Initially, the system receives a request for a set of due diligence questions (200) or a request for an RFP document relating to a medical topic. This may be seen as information that is inputted by a user and received by the system. In one embodiment, the request is received from one of the users via their communication device. In embodiments where the procurement process is for a medical product or is medical related, the request may include a medical topic. In other embodiments, the request may include a description of a requested service such as, but not limited to, a medical service, or critical feature of a non-medical solution.

In some embodiments, after receiving the request, the system may initialize and/or load all necessary parameters, models, and pre-indexed data for performing the method of generating a set of due diligence questions for use in creating an RFP for a procurement process. The parameters may relate to numerical thresholds for question generation, user-specific configurations, or semantic similarity thresholds for vector searches. The models may include one or more LLMs, summarization models, and embedding models.

In a specific example, the system 100 imports and initializes all required Python (or other scripting language) libraries and models and authenticates with a cloud service provider using appropriate credentials. This allows the system to access processing infrastructure and cloud-based models such as a general-purpose or general LLM and, if necessary, a medically specialized summarization model/LLM or a trained model/LLM.

In order to improve the processing of the method of the disclosure, the system may also load pre-existing datasets into memory. These datasets may include peer-reviewed scientific literature, previously approved due diligence questions (DDQs), and taxonomies of question categories derived from peer reviewed research or existing industry standard terminology. Alternatively, the system may communicate with external databases, data sources and/or external servers to retrieve this digital information or digital data.

In one specific implementation, all scientific or journal articles from one or more databases are loaded along with corresponding vector embeddings, precomputed using a semantic encoder. The system also loads and indexes example DDQs categorized by subdomain (e.g., clinical safety, performance metrics, etc.) for later use.

After initialization, the system enters its operational loop, which executes for each combination of product or service category and question category (based on the input from the user). Product and service categories may be derived from an established set of industry standards but are flexible to grow based on emerging trends found in publications, or as described in product supplier documentation.

The system processes the input to determine whether the input pertains to a product or service (201). In one embodiment, this is performed by passing a prompt to one of the LLMs, which classifies the user's input accordingly. This determination influences the phrasing and relevance of how the system handles other questions in the process. In one embodiment, the system expands the initial product or service input or input category term using the LLM to generate a related keywords and synonyms output. In one specific example, the related keywords and synonyms output is used to form two query vectors: one directly from the synonyms and another augmented with predefined terms relevant to due diligence.

After receiving the request, the system then retrieves a set of summaries associated with journal articles related to the medical topic (202). In one embodiment, the vectors generated in response to the related keywords and synonyms output are normalized and used to perform a nearest neighbor search against the set of retrieved journal articles (which may be seen as an embedded article dataset) to determine a set of top-ranked articles associated with the medical topic or input category. In some embodiments, other similarity metrics beyond cosine or Euclidean distance may be used, provided they capture proportional changes in semantic similarity.

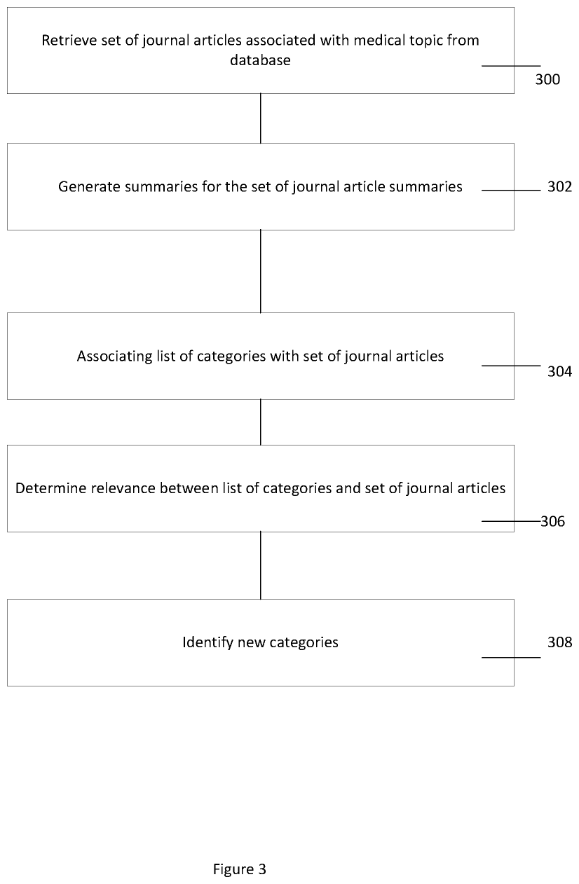

FIG. 3 is a flowchart showing one embodiment of journal article retrieval. In the embodiment of FIG. 3, the system communicates with a database such as, but not limited to, the peer-reviewed articles database to retrieve journal articles that have been stored in the database that relate to the medical topic (300). In some embodiments, the set of journal articles may be merged or matched with industry white papers. The system then processes the journal articles to generate summaries for the selected journal articles (302).

In one embodiment, the system transmits the retrieved journal articles to one of the external servers to process the journal articles which then creates or generates summaries or summarization prompts for each of the journal articles that are then sent back to the system and stored in the system database. In one embodiment, the summaries are generated by the external server 118 housing or storing a medical focused LLM. As discussed above, in other embodiments, the retrieval of the article summaries from the external server may be performed by the journal article summary generation module or may be performed by the CPU. Alternatively, the journal article summary or summaries may be performed by the journal summary generation module or by an LLM that is stored within the server 102 (or the journal article summary generation module 112d) to generate the summaries which can then be stored in the system database. In this embodiment, the medical focused LLM may be part of the system 100.

After the summaries are generated, the system may further process the journal articles by associating a list of predefined or emerging medical topics, medical product or medical service categories to the set of journal articles (304). In one embodiment, the system may access or communicate with the new medical devices categories database and/or the new medical products database to compare or associate the information stored in these database (medical device categories and/or new medical products categories) with the summaries of the journal articles or the journal articles themselves. Any selected category can then be received by the system and the association between the journal article and the selected category stored in the system. In some embodiments, the comparison is performed using a semantic search to determine which journal articles most closely match the medical topic. In one embodiment, this may be performed by the association module 112e. The system then determines a relevance of the set of journal articles with the retrieved categories (306). In some embodiments, the relevance may be determined by providing a score based on the comparison in (304) where journal articles that have a higher score represent journal articles that have more relevance to the medical topic. In some embodiments, this may be performed using a priority ranking methodology whereby language and focus of the journal article that more closely matches the medical topic are provided a higher ranking or score whereby the system selects a predetermined number of journal articles (in priority ranking order) are selected for use in the method of the disclosure. Based on the relevance determination, the system can then update due diligence question logic or a due diligence LLM. In a specific example, each journal article is assessed for relevance and summarized to extract clinical insights, which are retained only if they relate to the product or service under review.

The system may then identify new product or service categories (308) based on the relevance determination. In some embodiments, the selected journal articles or the journal articles determined to have a higher relevance to the medical topic are parsed to process the language within these journal articles to determine if there are new medical terms or categories that may relate to new medical products or services that are not part of the current listing of available medical products and medical services. In other embodiments, the system may parse the language of the selected journal articles or the journal articles determined to have a higher relevance to the medical topic to determine if the language relates to a taxonomy of current medical products and medical services stored in the system. These may then be added to the relevant databases (or to a medical product/medical service listing that is stored in a database) so that the system continuously updates the databases with the most recent information. In some embodiments, the new categories may also be associated with previously stored due diligence questions in order to improve the system's ability to generate a gold standard of DDQs for procurement.

After receiving the summaries, the system then retrieves a set of previously generated due diligence questions (204). In one embodiment, the set of previously generated due diligence questions may be stored in the database associated with the system. It will be understood that the retrieval of the set of previously generated due diligence questions (204) may be performed before or concurrent with the retrieval of the journal article summaries. In some embodiments, the set of previously generated due diligence questions includes a tag that indicates the medical topic to which the questions relate whereby this tag relates to the medical topic inputted by the user in (200).

In some embodiments, the system may generate content to support DDQ creation which may include, but is not limited to, a basic description of the medical topic or input category, relevant conditions and diseases it addresses, and a comprehensive list of dimensions or characteristics the system should consider. Prompts for this content are sent to one of the LLMs, either a specific LLM for this functionality or the single LLM performing all tasks of the disclosure.

The system then processes the combination of the set of previously generated due diligence questions and the journal article summaries (206). In some embodiments, to improve efficiency and relevancy, the system identifies and filters out irrelevant questions or question categories using exclusion logic derived from the medical product type and use case such that only relevant categories are retained for further processing. For each retained question category, the system may retrieve corresponding example questions and category descriptions. Based on configuration settings, the system determines the number of questions to be generated using system-derived versus user-provided data. A DDQ prompt is then created that incorporates the product category and its key characteristics; the summarized research findings or journal article summaries; example DDQs from the previously generated due diligence questions; and/or the target question category description.

In other embodiments, the processing of the combination of the set of previously generated due diligence questions and the journal article summaries is by a due diligence LLM or the question generation/refinement LLM or the general LLM.

The system then generates or receives a set of RFP or due diligence questions (208) based on the processing by or from the due diligence LLM or general LLM. As discussed above, if the due diligence LLM is part of the system, the system generates the due diligence questions and if the due diligence LLM is stored in an external server, the system may be seen as receiving or retrieving the due diligence questions although it is understood that the generation of the due diligence questions is part of the innovation. In embodiments, the due diligence questions are tagged with metadata such as, but not limited to, source provenance, associated clinical outcomes, and/or a timestamp.

In one embodiment, the generated DDQ prompt is passed to the LLM to generate a first set of draft DDQs. If user input was also provided (e.g., company-specific requirements or known product features), a second DDQ prompt is constructed with user-specific context, and an additional set of DDQs is generated. If there are two sets of DDQ generated, they may be combined and passed through a final refinement prompt so that the DDQ are concise, unbiased, and free of any proprietary terminology. If there is only one set of DDQ, these are passed by the system to the RFP generation module for generation of the RFP document based on the DDQ.

In some embodiments, the system may perform a fairness verification pass to evaluate the overall objectivity, neutrality, and relevance of the generated DDQ. In one embodiment, this is accomplished by comparing each question against a stored collection of historical manual edits and annotations that represent approved, equitable language patterns as reviewed by domain experts. The verification process checks for exclusion of brand names, consistency with the stated procurement goals or outcomes, and clarity of language. If deviations are detected, the system may automatically revise the questions or flag them for optional user review and feedback. The fairness verification is intended to promote alignment with value-based procurement principles and supports equitable vendor participation. The final set is then broken into individual items and stored in a collector variable.

The system reviews questions with the intent of producing outputs that are concise, unbiased, and free of any proprietary terminology. The system then generates an RFP for procurement based on the due diligence questions (210).

As the disclosure is designed to support collaborative, role-based interaction throughout the due diligence and procurement process, once the due diligence questions and relevant research summaries are generated by the system, the system may provide this information to subject matter experts to contribute their input, if desired. These experts may include clinicians (e.g., physicians, nurses), building operations personnel (e.g., facility engineers), information technology (IT) professionals, or other relevant stakeholders depending on the nature of the product or service being considered. This may be performed by digitally transmitting the due diligence questions and summaries to the experts for their input and then receiving their comments via a digital input.

Each user interaction, including edits, annotations, and approvals, may be tagged by role and assessed based on its contextual relevance to the question category under review by the system. For example, a clinician's feedback may be considered more relevant for questions concerning patient outcomes or safety, while IT professionals may provide more applicable input for technology integration or data security. This internal evaluation of role relevance is managed by the system and may influence both the final wording of due diligence questions and the ongoing refinement of model behavior.

While the system takes into account the role-based relevance of input when generating or revising questions, it always grounds its outputs in the most current and relevant peer-reviewed scientific research available at the time of generation. This ensures that user collaboration complements the evidence-based foundation of the system.

In order to avoid confusion with industry-standard procurement terminology, it is noted that the internal “weighting” described here refers to the system's prioritization of reviewer input based on their role's applicability to the question content which is distinct from the external weighting of questions in an RFP, which typically reflects the question's scoring value during vendor evaluation.

In some embodiments, the system maintains an audit trail of all contributions and offers customizable permission levels for reading, commenting, editing, and approving DDQs or RFP components. This collaborative approach supports interdisciplinary input, institutional consistency, and continuous learning across procurement cycles.

Role-based input may also play a role in the system's feedback loop architecture. The system tracks patterns in reviewer edits and approvals to inform the refinement of future DDQ outputs. When a particular role consistently adjusts language or clarifies outcomes for specific question categories, these patterns are stored and incorporated into the prompt engineering or fine-tuning pipeline, either globally or at the tenant level. Over time, this allows the system to anticipate institution-specific preferences and domain-specific improvements, enhancing the relevance and fairness of generated questions while reducing or minimizing manual rework.

In further embodiments, the method of FIG. 2 may be repeated for every iteration of the operational loop until all combinations of product or service category and question category are generated. In a specific implementation, results are exported as a CSV file to local or cloud-based storage. Additional logging or metadata (e.g., timestamps, article sources, or model versions) may also be stored to support traceability and version control.

An advantage of the disclosure is that it supports continuous iteration and can be configured to detect emerging product categories or unclassified trends through clustering of semantic embeddings or unsupervised topic modeling. This enables the system to adapt to novel inputs and evolving medical procurement needs. As discussed above, the disclosure is able to determine other or new terms (not input by the user) by parsing the journal articles to seek new terms or phrases. The system can then determine if these other or new terms are new medical topics or if they are associated or related to current medical topics.

In order to support secure, scalable, and responsive performance, the system infrastructure may include, but is not limited to, a combination of cloud and local resources. These may include execution on a cloud environment that includes a storage, computing and networking infrastructure. The system infrastructure may also a local computing node for pre-processing, data sanitation, and internal analytics. The system architecture may also include APIs/web interface to improve communication and feedback between the system and the users. The system architecture may also include security and verification so that information can be independently verified by matching with third-party data or regulatory databases.

Advantages of the disclosure include, but are not limited to, at least one of the generation of customized, outcomes-oriented DDQs in a much faster time frame than some current solutions; outputs are generated to reflect the most current available clinical research and procurement best practices; equitable solution support for novel or emerging vendors/products by framing criteria around outcomes, not brand name or historical precedent; adaptive question/requirement generation by incorporating internal procurement rules, certifications, and local regulations; helps users ask smarter questions that reveal deeper value beyond cost allowing for more innovative, value-weighted solutions; automatically identifies and incorporates emerging medical product/service categories that fit clinical outcomes; incorporates previous edits and feedback to improve future DDQs on an individual or corporation basis and includes mechanisms designed to identify and surface novel or emergent product or service categories.

By clustering retrieved literature based on topic modeling and analyzing new co-occurrence patterns in clinical outcomes, use cases, and terminology, the system can propose new product categories not previously present in its training data or in current lists of available medical products or medical services. These emergent categories may be presented to users for confirmation and added to the system's taxonomy, enabling continuous expansion of procurement horizons.

In one example, assume that the medical topic input by the user for an RFP is computed tomography (CT) scanners where one journal article is directed at photo-counting technology in CT scanners and a second journal article is directed at a comparison of technology X and photon-counting in CT scanners. Assuming this pattern continues in journal articles where technology X is regularly cited or parsed in journal articles selected for or associated to photo-counting in CT scanners, possibly in reference to diseases, or other solutions, the system can combine the phrase “technology X” with “photo-counting in CT scanners” or use the term “technology X” to create a new medical topic of technology X, or expand the taxonomy of an existing medical topic such as “Imaging Devices” to include technology X.

In another embodiment, the system may parse the set of journal articles to determine new medical terms within the set of journal articles where the new medical terms are seen as terms that are not currently listed in the current list of available medical products or medical services. The system then determines the number of times the new medical terms exist within the set of journal articles. If the number of times the new medical terms exist passes a predetermined threshold, the new medical terms are added to the list of available medical products or medical services. For those that do not pass a threshold, the new medical terms are associated with a current medical product or medical service. It is understood that the number of times the new medical terms appear in a journal article may be regularly updated or monitored whereby, over time, the new medical term may be transitioned into a new medical product or service that is available for selection.

The system is designed to support multi-tenant configurations, enabling multiple healthcare institutions, procurement organizations, or networks to operate independently within the same infrastructure. Each tenant maintains strict data isolation for organization-specific data including, but not limited to, custom DDQ language preferences, internal regulatory criteria, vendor relationships, and product specifications.

The disclosure enables the system to offer collective learning and continuously updated context for RFP creation while safeguarding sensitive or strategic organizational preferences. Each user or group of users can optionally choose whether to contribute to communal data sets while preserving proprietary configurations and preferences.

In some embodiments, procurement teams may also upload proposed capital equipment needs or anticipated future purchases, which the system can correlate with supplier product listings, DDQ generation frequency, and emerging research. These data points are then analyzed, such as via the analytics module 112g, to identify which product types, specifications, or clinical outcome criteria are drawing attention or scrutiny. For instance, suppliers may be able to see that ventilators emphasizing post-operative respiratory recovery are receiving heightened attention based on keyword intersections in DDQs and article summaries. In order to assist the suppliers, the system may provide metrics to the suppliers such as, but not limited to, aggregate views and interactions by product category or attribute; frequency of keyword matches with current research trends; market gaps identified by analyzing emerging related search trends, and the identification of common values, outcomes or features of related products; and/or competitive benchmarking (e.g., similarity to other listed solutions in terms of specifications or outcomes). This may assist both emerging and established vendors align their sales, product development, and marketing strategies to match the evolving needs of clinical buyers. At the same time, all insights are delivered in a privacy-protective manner—ensuring that no individual buyer, institution, or proprietary content is ever revealed.

The platform supports both static and adaptive model configurations. In one embodiment, one or more LLMs used for question generation and summarization are initialized from pre-trained base models (e.g., Gemini 2.0 Flash, Gemini 1.5 Pro and MedLM) and are further refined over time through feedback loops and fine-tuning mechanisms.

Fine-tuning may occur at multiple levels such as global fine-tuning where common feedback patterns and accepted edits across all users are incorporated to continuously enhance baseline model behavior, improving alignment with procurement best practices and clinical relevance or tenant-specific fine-tuning where each organization may activate a personalization layer in which model behavior is influenced by that organization's historic question preferences, regulatory constraints, and feedback data to enable outputs to reflect institutional language styles, documentation requirements, and clinical focus areas without affecting outputs for other tenants and/or prompt adaptation and embedding refinement where in addition to full model fine-tuning, the system supports adaptive prompting wherein prompt templates are updated based on observed improvements in clarity, utility, or performance such that embedding generation for semantic searches can also be updated periodically to account for evolving terminology and concept relationships within medical literature.

It would be appreciated by one of ordinary skill in the art that the system and components shown in the figures may include components not shown in the drawings. For simplicity and clarity of the illustration, elements in the figures are not necessarily to scale and are only schematic. It will be apparent to persons skilled in the art that a number of variations and modifications can be made without departing from the scope of the invention as described herein.

It is contemplated that any part of any aspect or embodiment discussed in this specification can be implemented or combined with any part of any other aspect or embodiment discussed in this specification, so long as such those parts are not mutually exclusive with each other.

It should be recognized that features and aspects of the various examples provided above can be combined into further examples that also fall within the scope of the present disclosure.

When used in this specification and claims, the terms “comprises” and “comprising” and variations thereof mean that the specified features, steps or integers are included. The terms are not to be interpreted to exclude the presence of other features, steps or components. Further, as used herein, the term “comprising” can mean “including.” Variations of the word “comprising”, such as “comprise” and “comprises,” have correspondingly varied meanings. Thus, for example, a composition “comprising” X may consist exclusively of X or may include one or more additional unrecited components. It will be understood that in embodiments which comprise or may comprise a specified feature or variable or parameter, alternative embodiments may consist, or consist essentially of such features, or variables or parameters. A reference to an element by the indefinite article “a” does not exclude the possibility that more than one of the elements is present, unless the context clearly requires that there be one and only one of the elements.

Additionally, the term “connect” and variants of it such as “connected”, “connects”, and “connecting” as used in this description are intended to include indirect and direct connections unless otherwise indicated. For example, if a first device is connected to a second device, that coupling may be through a direct connection or through an indirect connection via other devices and connections. Similarly, if the first device is communicatively connected to the second device, communication may be through a direct connection or through an indirect connection via other devices and connections.

The terms are not to be interpreted to exclude the presence of other features, steps or components. Further, the singular forms “a”, “an”, and “the” are intended to include the plural forms as well, unless the context clearly indicates otherwise.

The embodiments have been described above with reference to flow, sequence, and block diagrams of methods, apparatuses, systems, and computer program products. In this regard, the depicted flow, sequence, and block diagrams illustrate the architecture, functionality, and operation of implementations of various embodiments. For instance, each block of the flow and block diagrams and operation in the sequence diagrams may represent a module, segment, or portion of code, which comprises one or more executable instructions for implementing the specified action(s). In some alternative embodiments, the action(s) noted in that block or operation may occur out of the order noted in those figures. For example, two blocks or operations shown in succession may, in some embodiments, be executed substantially concurrently, or the blocks or operations may sometimes be executed in the reverse order, depending upon the functionality involved. Some specific examples of the foregoing have been noted above but those noted examples are not necessarily the only examples. Each block of the flow and block diagrams and operation of the sequence diagrams, and combinations of those blocks and operations, may be implemented by special purpose hardware-based systems that perform the specified functions or acts, or combinations of special purpose hardware and computer instructions.

Use of language such as “at least one of X, Y, and Z,” “at least one of X, Y, or Z,” “at least one or more of X, Y, and Z,” “at least one or more of X, Y, and/or Z,” or “at least one of X, Y, and/or Z,” is intended to be inclusive of both a single item (e.g., just X, or just Y, or just Z) and multiple items (e.g., {X and Y}, {X and Z}, {Y and Z}, or {X, Y, and Z}). The phrase “at least one of” and similar phrases are not intended to convey a requirement that each possible item must be present, although each possible item may be present. Further, in this disclosure, the term “or” is generally employed in its sense including “and/or” unless the content clearly dictates otherwise.

The invention may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied herein disclosed may be resorted to by those skilled in the art, and that such modifications and variations are considered to be within the scope of this invention.

What is claimed is:

1. A method for generating due diligence questions for procurement in the medical field comprising:
receiving a set of summaries of journal articles associated with a medical topic;
receiving a set of previously generated due diligence questions;
passing the set of summaries and the set of previously generated due diligence questions through a due diligence large language model (LLM);
receiving a set of due diligence questions from the due diligence LLM; and
generating a request for proposal (RFP) questionnaire based on the set of due diligence questions.

2. The method of claim 1 wherein receiving a set of summaries of journal articles comprises:
retrieving a set of journal articles; and
passing the set of journal articles through a document summary LLM to generate the set of summaries.

3. The method of claim 2 further comprising:
associating a list of predefined or emerging medical categories to the set of journal articles;
determining relevance of journal articles to the list of predefined or emerging medical product categories; and
updating due diligence question generation logic based on determined relevance.

4. The method of claim 3 further comprising:
identifying new product or service categories for the list of predetermined or emerging medical categories.

5. The method of claim 4 wherein identifying new product or service categories comprises:
parsing the set of journal articles to determine new medical terms within the set of journal articles;
determining a number of times the new medical terms exist within the set of journal articles;
determining if the number of times the new medical terms exist passes a predetermined threshold; and
adding the new medical terms that pass the threshold to the list of predetermined or emerging medical categories.

6. The method of claim 1 further comprising:
refining the set of due diligence questions by passing them through the due diligence LLM to enhance clarity and remove bias.

7. A system for generating a request for proposal (RFP) questionnaire for a medical topic comprising:
a RFP generation module including a due diligence large language model (LLM);
a journal article summary module including a document summary LLM;
a semantic encoder model for document embeddings;
a processor for communicating with the due diligence LLM and the document summary LLM;
wherein the processor passes a set of journal articles associated with the medical topic to the document summary LLM to generate a set of summaries;
wherein the processor passes the set of summaries and a set of previously generated due diligence questions to the due diligence LLM to generate a set of RFP questions.

8. The system of claim 7 further comprising:
a user interface for inputting organization-specific data and receiving customized outputs.

9. The system of claim 7 further comprising a comparison module for comparing the set of summaries with the set of previously generated due diligence questions.

10. The system of claim 7 further comprising an association module for associating the set of journal articles or set of summaries with a set of the medical topics.

11. The system of claim 7 further comprising an analytics module for processing and providing analytical insight to users.

12. The system of claim 7 further comprising a new topic generation module for generating new medical topics.

13. The system of claim 7 wherein the due diligence LLM and the document summary LLM are a single LLM.
